(19) United States
(12) Patent Application Publication
(10) Pub. No.: US 2026/0124444 A1
(43) Pub. Date: May 7, 2026

(54) MODIFYING PH OF TISSUE TO REVERSE IMMUNOSUPRESSION

(71) Applicant: Virginia Tech Intellectual Properties, Inc., Blacksburg, VA (US)

(72) Inventors: Rafael V. Davalos, Aptos, CA (US); Zaid Samer Salameh, Blacksburg, VA (US); Kenneth N. Aycock, Blacksburg, VA (US)

(21) Appl. No.: 19/120,676

(22) PCT Filed: Oct. 11, 2023
(86) PCT No.: PCT/US23/76626

§ 371 Date: Apr. 11, 2025

Related U.S. Application Data
(60) Provisional application No. 63/414,942, filed on Oct. 11, 2022.

(51) Int. Cl. A61N 1/32; A61N 1/04

(52) U.S. Cl. CPC A61N 1/327; A61N 1/0412; A61N 1/0476

(57) ABSTRACT

Embodiments of the present invention include methods of targeting acidosis (low pH) within the tumor microenvironment (TME) through the use of cathodic electrochemical reactions (CER). Low pH is oncogenic by supporting immunosuppression. Electrochemical reactions create local pH effects when a current passes through an electrolytic substrate such as biological tissue. Electrolysis has been used with electroporation (destabilization of the lipid bilayer via an applied electric potential) to increase cell death areas. However, the regulated increase of pH through only the cathode electrode has been ignored as a possible method to alleviate TME acidosis, which could provide substantial immunotherapeutic benefits. Here, ex vivo modeling shows that CERs can intentionally elevate pH to an anti-tumor level and that increased alkalinity promotes activation of naïve macrophages. Embodiments of the invention include pairing CER treatment protocols with existing electric field-based cancer therapies or use as a stand-alone therapy.

### CROSS-REFERENCE TO RELATED APPLICATIONS

This application relies on the disclosure of and claims priority to and the benefit of the filing date of U.S. Provisional Application No. 63/414,942 filed Oct. 11, 2022, which is hereby incorporated by reference herein in its entirety.

### BACKGROUND OF THE INVENTION

### Field of the Invention

The present invention relates to the field of electrical energy based treatments.

### Description of Related Art

Acidity (low pH) is an oncogenic characteristic of the tumor microenvironment, supporting immunosuppression and tumor expansion (Huber, V. et al. Cancer acidity: An ultimate frontier of tumor immune escape and a novel target of immunomodulation. Semin. Cancer Biol. 43:74-89, 2017). Low pH arises from increased production of lactate and hydrogen ions in malignant cells that increasingly rely on aerobic glycolysis (Warburg effect) (Liberti, M. V., and J. W. Locasale. The Warburg Effect: How Does it Benefit Cancer Cells? Trends Biochem. Sci. 41:211-218, 2016). At a lower pH, T lymphocytes and natural killer (NK) cell function decrease and cells may become apoptotic (Calcinotto, A. et al. Modulation of microenvironment acidity reverses anergy in human and murine tumor-infiltrating T lymphocytes. Cancer Res. 72:2746-2756, 2012; Loeffler, D. A. et al. Natural killer-cell activity under conditions reflective of tumor micro-environment. Int. J. Cancer 48:895-899, 1991). Conversely, immuno-suppressive cells (regulatory T cells) activate (Watson, M. J. et al. Metabolic support of tumour-infiltrating regulatory T cells by lactic acid. Nature 591:645-651, 2021) and tumor-associated macrophages (TAMs) transform into a pro-tumor phenotype (El-Kenawi, A. et al. Acidity promotes tumour progression by altering macrophage phenotype in prostate cancer. Br. J. Cancer 121:556-566, 2019). Therefore, tumor acidity is a critical regulator of cancer immunity that orchestrates both local and systemic immunosuppression (Damgaci, S. et al. Hypoxia and acidosis: immune suppressors and therapeutic targets. Immunology 154:354-362, 2018), providing a need for therapeutic targets.

Previous studies have targeted tumor pH using oral buffers (sodium bicarbonate) to elevate the TME pH and encourage immune cell infiltration (Pilot, C., A. Mahipal, and R. J. Gillies. Buffer Therapy→Buffer Diet. J. Nutr. Food Sci. 08: 2018). While effective in preventing metastases, sodium bicarbonate therapy does not address the primary tumor when used as a monotherapy (Robey, I. F. et al. Bicarbonate Increases Tumor pH and Inhibits Spontaneous Metastases. Cancer Res. 69:2260-2268, 2009). Recently, a combinatorial therapy of ethanol ablation (to treat the primary tumor) in conjunction with oral sodium bicarbonate (to elevate tumor pH) and cyclophosphamide (to deplete regulatory T cells) proved effective in treating the primary tumor and in preventing metastases (Nief, C. A. et al. Targeting Tumor Acidosis and Regulatory T Cells Unmasks Anti-Metastatic Potential of Local Tumor Ablation in Triple-Negative Breast Cancer. Int. J. Mol. Sci. 23:8479, 2022).

One neoplasm associated with chronic inflammation (and an oncogenic TME) is Hepatocellular Carcinoma (HCC) (Coussens, L. M., and Z. Werb. Inflammation and cancer. Nature 420:860-867, 2002), which leads to sustained changes in both the innate hepatic immune response and systemic immune cell infiltration (Ringelhan, M. et al. The immunology of hepatocellular carcinoma. Nat. Immunol. 19:222-232, 2018). Surgical resection (transplant or partial hepatectomy) currently provides the best clinical strategy to treat HCC patients but can be limited by late diagnosis, tumor size and/or location, underlying pathology, and lack of organs for transplant (Balogh, J. et al. Hepatocellular carcinoma: a review. J. Hepatocell. Carcinoma 3:41-53, 2016). Although thermal ablation (radiofrequency and microwave ablation (RFA/MWA)) has emerged as a viable alternative to resection for liver neoplasms (Llovet, J. M. et al. Locoregional therapies in the era of molecular and immune treatments for hepatocellular carcinoma. Nat. Rev. Gastroenterol. Hepatol. 18:293-313, 2021), the indiscriminate tissue damage arising within the ablative zone can lead to challenges when ablating tumors located near critical structures.

Irreversible electroporation (IRE) has emerged as an alternative to thermal ablation (Davalos, R. V. et al. Tissue Ablation with Irreversible Electroporation. Ann. Biomed. Eng. 33:223-231, 2005). With IRE systems a high voltage electrical potential is delivered in short pulses (80-100 μs) across the target region between appropriately placed electrodes leading to the formation of nanodefects in the lipid bilayer of cells within the electric field. These nanodefects can lead to loss of homeostasis and induce cell death pathways (Aycock, K. N., and R. V. Davalos. Irreversible Electroporation: Background, Theory, and Review of Recent Developments in Clinical Oncology. Bioelectricity 1:214-234, 2019). Unlike thermal ablation, IRE selectively induces cell death within the ablation zone without damage to the underlying tissue architecture, preventing damage to structures such as blood vessels, ducts, and nerves (Maor, E. et al. The Effect of Irreversible Electroporation on Blood Vessels. Technol. Cancer Res. Treat. 6:307-312, 2007; Phillips, M. et al. Nonthermal Irreversible Electroporation for Tissue Decellularization. J. Biomech. Eng. 132:091003, 2010). Notably, due to its non-thermal cell death mechanism, IRE treatment initiates a robust anti-tumor response by preserving antigen presentation (Beitel-White, N. et al. Davalos. Real-time prediction of patient immune cell modulation during irreversible electroporation therapy. Sci. Rep. 9:17739, 2019; Imran, K. et al. Exploration of Novel Pathways Underlying Irreversible Electroporation Induced Anti-Tumor Immunity in Pancreatic Cancer. Front. Oncol. 12:1, 2022; Ringel-Scaia, V. M. et al. High-frequency irreversible electroporation is an effective tumor ablation strategy that induces immunologic cell death and promotes systemic anti-tumor immunity. EBioMedicine 44:112-125, 2019).

Electrochemical reactions that occur proximal to the electrodes in IRE treatments alter the tissue's pH immediately around the cathode (alkaline) and anode (acidic) (Turjanski, P. et al. The Role of pH Fronts in Reversible Electroporation. PLOS ONE 6: e17303, 2011). Others have exploited electrolysis to generate toxic byproducts to increase ablation volumes by taking advantage of the reversible electroporation regime (Klein, N. et al. Single exponential decay waveform; a synergistic combination of electroporation and electrolysis (E2) for tissue ablation. PeerJ 5: e3190, 2017; Phillips, M. et al. Tissue ablation by a synergistic combination of electroporation and electrolysis delivered by a single pulse. Ann. Biomed. Eng. 44:3144-3154, 2016). In such cases, the electrochemical reactions are maximized through monophasic, long (or DC), low voltage pulses, or high-charge exponentially decaying pulses (Rubinsky, L. et al. Electrolytic Effects During Tissue Ablation by Electroporation. Technol. Cancer Res. Treat. 15:NP95-NP103, 2016). Importantly, electrolytic electroporation uses bipolar geometries in which both the cathode and anode contribute to the formation of electrolytic byproducts. There remains a need for methods of administering electrical pulses in a manner to elevate the pH and reduce the immunosuppressive nature of the TME.

### SUMMARY OF THE INVENTION

Embodiments of the invention include methods of elevating or lowering local pH through administration of electrical pulses capable of causing electrochemical reactions (e.g., CER, AER) in the target area. Other embodiments of the present invention include combinatorial therapy comprising a first treatment protocol to target the primary tumor (e.g., IRE and/or H-FIRE) and a second treatment protocol configured to change the local pH (such as elevate the local pH through electrochemical reactions to support the anti-tumor immune response).

Included in embodiments is Aspect 1, which is a method of providing electrical energy therapy comprising: applying electrical pulses to a target region sufficient to induce H-FIRE and/or IRE; and applying electrical pulses insufficient to induce electroporation, and sufficient to change the pH of the target region; and optionally measuring the change in pH, wherein optionally the pH changing pulses comprise 1) a set of CER pulses delivered through high frequency, monophasic pulsing where the pulse width is very short (1-100 μs) (or any range in between) and there are delays between pulses (1 μs to 1 s) (or any range in between) until the total on time has reached 10-1,000 seconds (or any range in between), delivered at voltages between 10 V and 250 V (or any range in between) and currents between 0.5 A and 10 A (or any range in between), and/or 2) a set of CER pulses comprising DC pulses (1 second to 1000 seconds) (or any range in between). In embodiments, a DC pulse can be left on without any delays for several hundred seconds to create the pH change. This can be delivered at voltages between 10 and 250 V (or any range in between) and currents between 0.5 A and 10 A (or any range in between). DC pulses can also be delivered with pulse lengths of 10 ms, 25 ms, 50 ms, 75 ms, 100 ms, or 200 ms (or any range in between).

Aspect 2 is a method of Aspect 1 comprising: delivering a first set of electrical pulses comprising: IRE pulses of up to 500 electrical pulses (such as in the range of 100-300 pulses), at a voltage ranging from 1,000 V to 5,000 V (such as in the range of 1,000-3,000 V), to a target region (such as a tissue or tumor), wherein the IRE pulses have a length of from 50-200 microseconds (such as in the range of 50-100 microseconds) and the IRE pulses are delivered at a rate of 1 Hz (with one second between the start of each IRE pulse); and/or H-FIRE pulses of up to 500 electrical pulses (e.g., in the range of 100-300 pulses), at a voltage ranging from 1,000-5,000 V (such as in the range of 1,000-3,000 V), to a target region (such as a tissue or tumor), wherein the H-FIRE pulses have a length of from 1-10 microseconds and are delivered at a rate of 1 Hz (with one second between the start of each H-FIRE pulse); and delivering a second set of electrical pulses capable of increasing or decreasing pH by 1-2 units within the target region (e.g., tissue or tumor); wherein the first set of pulses is delivered before, after or during the second set of pulses.

Aspect 3 is the method of Aspect 1 or 2, wherein: the first set of electrical pulses are monopolar IRE pulses.

Aspect 4 is the method of any of Aspects 1-3, wherein the second set of electrical pulses are DC pulses or high frequency, monophasic pulses.

Aspect 5 is the method of any of Aspects 1-4, comprising: increasing the pH of a tumor from acidic to homeostatic by applying the second set of pulses as a select number of a select length of DC pulses or high-frequency, monophasic pulses for a select period of time sufficient to raise the pH; and reversing immunosuppression of a tumor microenvironment.

Aspect 6 is the method of any of Aspects 1-5, wherein: the DC pulses have a length of from 1-1,000 seconds, are applied for a total on time in the range of 10-1,000 seconds, and are applied at a voltage ranging from 10-250 V and a current in the range of 0.5 A to 10 A.

Aspect 7 is the method of any of Aspects 1-6, comprising: applying the second set of pulses in a manner to increase pH and elevate an immune response, in a manner to decrease pH and suppress inflammatory response, or in a manner to decrease pH and suppress inflammatory response.

Aspect 8 is the method of any of Aspects 1-7, wherein: the first set of electrical pulses are IRE pulses: in the range of 100-300 pulses; delivered at a voltage in the range of 1,000-3,000 V; and having a length of from 50-100 microseconds; and the second set of electrical pulses: comprises monophasic, high-frequency pulses of from 1-100 microseconds with delays between pulses of from 1-100 microseconds, or comprises DC pulses with a length of from 1-1,000 seconds is applied for a total on time in the range of 10-1,000 seconds; is applied at a voltage of 10-250 V and a current in the range of 0.5-10 A.

Aspect 9 is the method of any of Aspects 1-8, wherein: the first set of electrical pulses: comprises a number of H-IRE pulses in the range of 100-300 pulses; is applied at a voltage of 1,000-3,000 V; and the second set of electrical pulses: comprises monophasic, high-frequency pulses of from 1-100 microseconds with delays between pulses of from 1-100 microseconds, or comprises DC pulses with a length of from 1-1,000 seconds; is applied for a total on time in the range of 10-1,000 seconds; is applied at a voltage of 10-250 V and a current in the range of 0.5-10 A.

Aspect 10 is the method of any of Aspects 1-9, wherein: the second set of pulses is delivered in a manner to increase the pH from acidic to basic.

Aspect 11 is the method of any of Aspects 1-10, wherein the second set of pulses: have a pulse length of microseconds to 10 s of seconds; are applied for 10-100 pulses; and are applied at a voltage in the range of 10-100 V.

Aspect 12 is the method of any of Aspects 1-11, wherein the change in pH is a change of 1-2 units, i.e., an increase of 1-2 units or a decrease of 1-2 units.

Aspect 13 is the method of any of Aspects 1-12, wherein the electrical pulses are AC pulses, DC pulses, or a combination of AC and DC pulses.

Aspect 14 is the method of any of Aspects 1-13, wherein: applying a plurality of electrical pulses to a tissue, wherein the plurality of electrical pulses is capable of causing electroporation and electrolysis of the tissue.

Aspect 15, which can be used alone or together with any of Aspects 1-14, is a method comprising: administering a plurality of electrical pulses to a tumor, wherein the plurality of electrical pulses is administered in a manner capable of: changing the pH of the tumor microenvironment; and/or causing a desired immune response; and/or improving immune cell infiltration; and/or controlling immune cell phenotype.

Aspect 16, which can be used alone or together with any of Aspects 1-15, is a method comprising: connecting one or more or multiple probes, such as two or more probes, to the cathode of a pulse generator; connecting a ground electrode (such as an external, plate or surface electrode) to the anode; and delivering a plurality of electrical pulses to a treatment area; wherein at least one pulse of the plurality of electrical pulses is capable of causing irreversible electroporation of at least some cells in the treatment area; and wherein at least one pulse of the plurality of electrical pulses is capable of changing the pH of the treatment area.

Aspect 17 is the method of any of Aspects 1-16, wherein the ground electrode is a distant ground electrode that is a surface electrode.

Aspect 18 is the method of any of Aspects 1-17, wherein at least one pulse of the plurality of electrical pulses is applied at a voltage capable of creating a pH change, but below a voltage threshold capable of nerve excitation and/or irreversible electroporation.

Aspect 19, which can be used alone or together with any of Aspects 1-18, is a method of providing electrical energy therapy comprising: applying electrical pulses to a target region sufficient to induce H-FIRE or IRE; and applying electrical pulses sufficient to change the pH of the target region.

Aspect 20, which can be used alone or together with any of Aspects 1-19, is a method comprising: delivering a first set of up to 500 electrical pulses, at a voltage ranging from 1,500 V to 5,000 V, to a tissue or tumor, wherein the pulses have a length of from 50-200 microseconds and the pulses are delivered at a rate with one second between each pulse; and delivering a second set of up to 100 electrical pulses, at a voltage ranging from 1-50 V, to the tissue or tumor, wherein the pulses have a length of from 50-200 microseconds, and allowing a local pH increase of 1-2 units within the tissue or tumor.

Aspect 21 is the method of any of Aspects 1-20, wherein: the first set of electrical pulses induces IRE in the tissue or tumor.

Aspect 22 is the method of any of Aspects 1-21, wherein the second set of electrical pulses are DC pulses.

Aspect 23, which can be used alone or together with any of Aspects 1-22, is a method of reversing immunosuppression of a tumor microenvironment comprising: increasing the pH of a tumor from acidic to homeostatic by applying a select number of a select length of DC pulses for a select period of time sufficient to raise the pH.

Aspect 24 is the method of any of Aspects 1-23, wherein: the DC pulses have a length of from 50-200 microseconds, are applied in an amount ranging from 2-50 pulses, and are applied at a voltage ranging from 2-50 V.

Aspect 25, which can be used alone or together with any of Aspects 1-24, is a method comprising: performing an ECT or GET treatment; and applying DC pulses in a manner to increase pH and elevate an immune response.

Aspect 26, which can be used alone or together with any of Aspects 1-25, is a method comprising: performing cardiac ablation; and applying DC pulses in a manner to decrease pH and suppress inflammatory response.

Aspect 27, which can be used alone or together with any of Aspects 1-26, is a method of suppressing inflammation comprising: applying DC pulses in a manner to decrease pH and suppress inflammatory response.

Aspect 28, which can be used alone or together with any of Aspects 1-27, is a method comprising: applying DC pulses in a manner to increase pH from acidic to basic, thereby i) changing macrophage phenotype from M2 to M1 and vice versa and/or ii) changing T cell phenotype from regulatory to cytotoxic and vice versa.

Aspect 29 is the method of any of Aspects 1-28, wherein the DC pulses: have a pulse length of microseconds to 10 s of seconds; are applied for 10-100 pulses; and are applied at a voltage of 10-100 V.

Aspect 30 is the method of any of Aspects 1-29, wherein the change in pH is a change of 1-2 units, i.e., an increase of 1-2 units or a decrease of 1-2 units.

Aspect 31 is the method of any of Aspects 1-30, wherein the electrical pulses are AC pulses, DC pulses, or a combination of AC and DC pulses.

Aspect 32, which can be used alone or together with any of Aspects 1-31, is a method comprising: applying a plurality of electrical pulses to a tissue, wherein the plurality of electrical pulses is capable of causing electroporation and electrolysis of the tissue.

Aspect 33, which can be used alone or together with any of Aspects 1-32, is a method comprising: administering a plurality of electrical pulses to a tumor, wherein the plurality of electrical pulses is administered in a manner capable of: changing the pH of the tumor microenvironment; and/or causing a desired immune response; and/or improving immune cell infiltration; and/or controlling immune cell phenotype.

Aspect 34, which can be used alone or together with any of Aspects 1-33, is a method comprising: connecting multiple probes, such as two or more probes, to the cathode of a pulse generator; connecting a distant ground electrode to the anode; and delivering a plurality of electrical pulses to a treatment area; wherein at least one pulse of the plurality of electrical pulses is capable of causing irreversible electroporation of at least some cells in the treatment area; and wherein at least one pulse of the plurality of electrical pulses is capable of changing the pH of the treatment area.

Aspect 35 is the method of any of Aspects 1-34, wherein the distant ground is a surface electrode.

Aspect 36 is the method of any of Aspects 1-35, wherein at least one pulse of the plurality of electrical pulses is applied at a voltage capable of creating a pH change, but below a voltage threshold capable of nerve excitation and/or irreversible electroporation.

### BRIEF DESCRIPTION OF THE DRAWINGS

The accompanying drawings illustrate certain aspects of implementations of the present disclosure, and should not be construed as limiting. Together with the written description the drawings serve to explain certain principles of the disclosure.

FIG. 1 is an illustration showing use of IRE treatment to generate a local alkaline environment.

FIG. 2A is an illustration showing multiple needle electrodes serving as the cathodes and anodes in a traditional IRE procedure.

FIG. 2B is an illustration showing a set of needle electrodes acting as the cathodes and a surface electrode acting as the anode according to an embodiment of the invention.

FIG. 3A is a graph showing the area of increased pH vs. applied current according to an embodiment of the invention.

FIG. 3B is a graph showing the area of increased pH vs. voltage for a pulse delivered at a constant current (0.100 Amps).

FIG. 3C is a graph showing the area of increased pH vs. pulse width.

FIG. 3D is an illustration showing a tissue phantom treated with a 1 second 10 V pulse (blue region indicates a pH increase >1.0).

FIGS. 4A-I are illustrations showing central regions of alkalinity in treated tissue phantoms. The IRE pulse protocol (FIGS. 4A-C) comprised 200 IRE pulses (100 μs pulse length, 1000 V). The pH-IRE pulse protocol (FIGS. 4D-F) comprised 100 IRE pulses and 100 pH pulses. The pH-IRE and surface electrode pulse protocol (FIGS. 4G-I) comprised administration of 100 IRE pulses, followed by connection of all needles to the cathode and connection of the surface electrode to the anode, followed by the 100 pH pulses.

FIG. 5A is a graph showing the effect of increasing pH on THP-1 macrophage CD14 expression detected in viable THP-1 cells using flow cytometry analysis. p=0.1678 (ns).

FIG. 5B is a graph showing the effect of increasing pH on THP-1 macrophage cell viability. * p<0.01 versus all other pH levels tested, n=6 independent experiments.

FIG. 5C is a graph showing the effect of increasing pH on THP-1 macrophage CD206 expression (M2 activation marker) detected in viable THP-1 cells using flow cytometry analysis. ** p<0.0001 vs pH 7.5 and pH 6.5, *p<0.01 vs pH 6.5, n=6 independent experiments.

FIG. 5D is a graph showing the effect of increasing pH on THP-1 macrophage CD80 expression (M1 activation marker) detected in viable THP-1 cells using flow cytometry analysis. *p<0.0001 vs pH 7.5 and pH 6.5, p=0.9094 (ns) vs pH 6.5, n=6 independent experiments.

FIGS. 6A-B are graphs showing the viability and activation of THP-1 macrophages when co-cultured with HEPG2 cells treated with varying combinations of HFIRE, CERs, and AERs.

FIGS. 7A-B are drawings showing probe placement for IRE (FIG. 7A) and CER (FIG. 7B) liver experiments.

FIG. 7C is an image showing special resolution of pH measurements collected in 2 mm increments from the center point of the electrode tip immediately following IRE and CER treatment.

FIG. 8A is an image showing a tissue (liver) slice following IRE delivery highlighting a necrotic (darker) center and outer transition zone.

FIG. 8B is an image showing changes in pH zones measured experimentally and superimposed across the tissue slice.

FIG. 8C is a graph showing the change in pH in tissue over time following IRE delivery measured at the point indicated with a star in FIG. 8B. n=3 independent experiments.

FIG. 9A is a graph showing pH changes over time post pH-IRE, traditional IRE, and High Frequency IRE (HFIRE) treatment.

FIG. 9B is a graph showing the change in pH vs. increasing pulse length for the pH-IRE treatment.

FIG. 9C is an illustration showing the pH change in an ex-vivo porcine liver using two needles connected to a cathode and a distant grounding ring for the anode.

FIG. 9D is an illustration showing the pH change in an ex-vivo porcine liver using one needle connected to the cathode and a second needle connected to the anode.

FIG. 10 is a drawing showing various pulse waveforms.

### DETAILED DESCRIPTION OF VARIOUS EMBODIMENTS OF THE INVENTION

Electroporation-based treatments such as Irreversible Electroporation (IRE) are becoming increasingly popular for the treatment of solid tumors due to their targeted effects on cells and have recently been identified to stimulate a substantial immune response, improving treatment success (Davalos, R. V. et al. Tissue Ablation with Irreversible Electroporation. Ann Biomed Eng. 2005; 33 (2):223-231). After treatment, the cellular debris from necrotic tissue is flush with inflammatory cytokines that signal for immune cell recruitment. Recent literature suggests IRE may produce an increased immune response when compared to other focal ablation modalities (Beitel-White N. Real-time prediction of patient immune cell modulation during irreversible electroporation therapy. Scientific Reports (2019) 9:17739). Although the mechanisms for the increased immune response are not fully understood, it is hypothesized to correlate with IRE's cell death mechanism. Jiang et al. propose a lethal membrane disruption process from electrical pulsing, which may result in elevated concentrations of inflammatory signaling molecules found in necrotic tissue as compared to the application of extreme temperatures seen in other ablation modalities (Jiang, C. et al. A Review of Basic to Clinical Studies of Irreversible Electroporation Therapy. IEEE Trans Biomed Eng. 2015; 62 (1): 4-20). While electroporation treatment plans prioritize cell death, herein a method is described in which pulsed-field ablation strategies can be leveraged to further enhance the downstream effects of the increased immune response from IRE.

Electrochemical reactions are a side effect of voltages being applied across tissues. This is primarily a result of electrolytic reactions occurring around the electrode-medium interface. A common reaction is the electrolysis of water forming hydrogen and oxygen gas. The electrolysis of water forms basic and acidic by-products at the cathode and anode respectively, although it should be noted chemical species are also dependent on electrode material. The choice of inert electrodes, such as platinum or palladium, provide the ability to generate pH change through electrolysis with minimal chemical species produced as byproducts. Alternatively, aluminum, steel, or silver chloride electrodes could be leveraged. At the cathode, water is reduced to hydrogen gas and produces hydroxide ions, ultimately increasing the tissue's pH. Conversely, at the anode water is oxidized to oxygen gas and produces hydrogen ions, decreasing the local pH.

$$2 {H}_{2} O + 2 e -= {H}_{2} ( g a s ) + 2 OH - ( base ) \qquad ( Equation 1 )$$

$$2 {H}_{2} O = 4 e - + {O}_{2} ( g a s ) + 4 H + ( acid ) \qquad ( Equation 2 )$$

Some groups have studied these effects to minimize the role of pH fronts in their desired treatment, while others use the electrolytic effects to increase the ablation area. There is an understanding that current plays an important role in generating electrochemical effects, but these effects have not been studied to intentionally increase the TME to homeostatic levels.

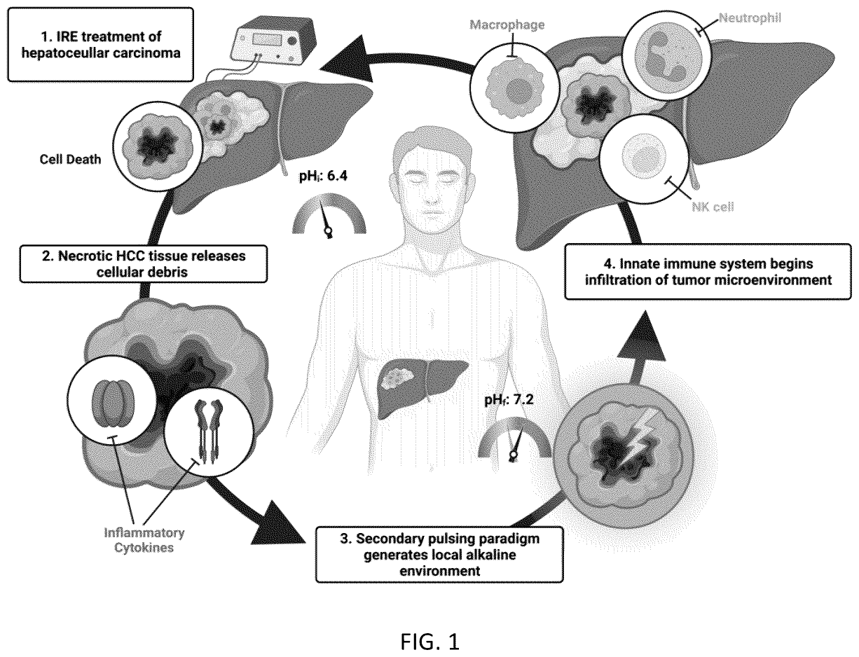

Thus, according to methods of the present invention, a secondary pulsing paradigm is employed after the administration of irreversible electroporation to generate a local alkaline environment and encourage immune cell infiltration (FIG. 1). First, pulsing parameters most strongly governing local pH in agar tissue phantoms were investigated. After determining the desired pulse parameters, this novel paradigm was administered in ex vivo porcine liver to determine the magnitude and decay rate of these pH changes. Proper parameter selection resulted in a substantial (>1.0) change in pH that was sustained for several hours after treatment, which may result in the improved clinical success of electroporation-based treatments.

Embodiments of the present invention include methods of delivering electrical pulses to control pH magnitude, decay rate, and area of impact. To identify the optimal pulse paradigm for the intended pH changes, a tissue phantom dyed with a pH indicator was used to conveniently show areas of an appreciable pH change (>1.0). Thereafter, the pulse parameters are translated into an ex vivo porcine liver model. As pH change cannot accessibly be visualized within a liver, here a pH electrode was utilized to provide point measurements on the surface of the liver after treatment.

Tissue phantom preparation: 1% weight/volume agar solution was prepared with DI water and 1/10 ratio of bromothymol blue. Bromothymol blue is a pH indicator that turns from green (pH 7.0) to dark blue (pH>7.6) or yellow (pH<6.0). The solution was boiled, stirred, and split into groups with varying NaCl concentrations (0.1-1.0%) for conductivity control. The solutions were pipetted into 150 mm petri dishes (50 mL per well) and allowed to solidify for 24 hours. Treatments were applied in each well using an ECM 830 Square Wave Electroporator (Harvard Apparatus, Holliston, MA). Current and voltage readings were collected using a wideband current monitor (Pearson Electronics, Palo Alto, CA) connected to an oscilloscope (Wavesurfer 3024z, Teledyne LeCroy, Chesnut Ridge, NY). Treatment was applied using a custom needle-grounding ring electrode configuration. Photographs were captured and ImageJ (NIH, Bethesda, MD) was used to quantify pH disturbance area. The pH disturbance areas were defined as areas with a pH greater than 7.6 after treatment, with the initial pH being 6.6.

### Investigation of Current, Voltage, and Electrical Dose

Irreversible electroporation treatment planning involves inputting voltage, pulse duration, pulse number, and frequency of pulses. Typical treatment involves 80-100 μs pulses at 1 Hz (Aycock, K. N., and R. V. Davalos. Irreversible Electroporation: Background, Theory, and Review of Recent Developments in Clinical Oncology. Bioelectricity 1:214-234, 2019), while voltage and pulse number can depend on the size of the tumor, the geometry of electrodes, and other clinical considerations. In these tests, current, electrolyte concentration, voltage, and pulse duration were manipulated by following Ohm's law to change desired variables. There is an understanding that the change in pH is closely related to the current, so in the first test, current was increased while keeping voltage and pulse duration constant. To increase current while maintaining a constant voltage, the resistance must be effectively changed to stay within the considerations of Ohm's law. This was done by adding NaCl to the agar, increasing the effective current at constant voltages. Next, the effect of increasing voltage on pH while holding current constant was investigated. This was also done by adding known amounts of NaCl to the agar, allowing the conductivity and resistance to be known. Following Ohm's law, voltage can be increased without changing current by determining resistance. Thereafter pulse duration was increased without changing the electrical dose.

$$Dose = {E}^{2} * η * τ \qquad ( Equation 3 )$$

Where E is the electric field, η is the pulse number, and τ is the pulse duration. The electric field can be substituted with Voltage (V) in the modified equation because the electric field-voltage relationship is dependent on geometry which was held constant. The pulse number was also held constant, so it was removed.

$$Modified Dose Equation = {V}^{2} * τ \qquad ( Equation 4 )$$

With the modified dose equation, an inversely squared relationship is observed between voltage and pulse duration.

In Tables 1 and 2, Pulse width (10 ms), pulse number (100), and frequency (1 Hz) were held constant while voltage or current was increased in the NaCl-containing phantoms. A single pre-pulse (10 V, 50 μs length) was delivered to each phantom to determine initial resistance and Ohm's law used to determine the change in voltage (or current) required to deliver constant current (or maintain a predetermined voltage). In Table 3, the dose (Equation 4) was held constant while voltage and current decreased and pulse width increased.

TABLE 1
Increasing Current (constant voltage and pulse width)

| σ(S/m) | Voltage (V) | Current (A) | η# | τ (ms) |
|---|---|---|---|---|
| 0.1 | 100 | 0.05 | 99 | 10 |
| 0.2 | 100 | 0.1 | 99 | 10 |
| 0.3 | 100 | 0.2 | 99 | 10 |
| 0.4 | 100 | 0.35 | 99 | 10 |
| 0.5 | 100 | 0.5 | 99 | 10 |

TABLE 2
Increasing Voltage (constant current and pulse width)

| σ(S/m) | Voltage (V) | Current (A) | η# | τ (ms) |
|---|---|---|---|---|
| 0.5 | 10 | 0.2 | 99 | 10 |
| 0.4 | 100 | 0.2 | 99 | 10 |
| 0.3 | 350 | 0.2 | 99 | 10 |
| 0.2 | 700 | 0.2 | 99 | 10 |
| 0.1 | 1000 | 0.2 | 99 | 10 |

TABLE 3
Increasing Pulse Duration (constant electrical dose)

| σ(S/m) | Voltage (V) | Current (A) | η# | τ (ms) |
|---|---|---|---|---|
| 0.2 | 1000 | 0.4 | 99 | 0.1 |
| 0.2 | 447 | 0.15 | 99 | 0.5 |
| 0.2 | 316 | 0.1 | 99 | 1 |
| 0.2 | 141 | 0.07 | 99 | 5 |
| 0.2 | 100 | 0.04 | 99 | 10 |
| 0.2 | 31 | 0.02 | 99 | 100 |
| 0.2 | 10 | 0.009 | 99 | 1000 |

### Effects of Probe Geometry

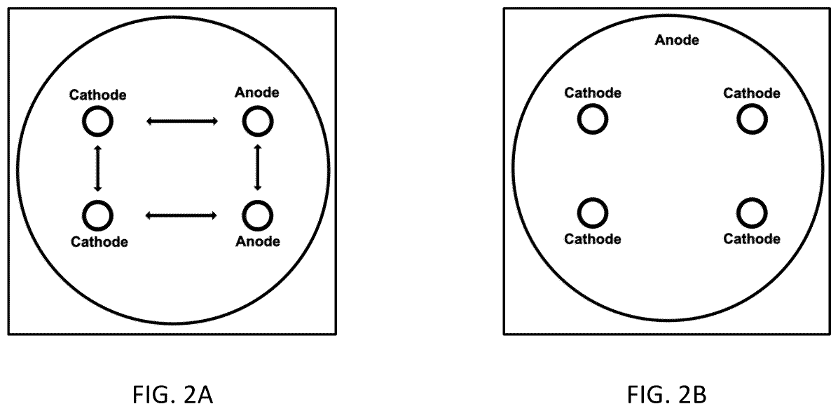

Clinically, IRE treatment is delivered by inserting at least two needle electrodes into the tumor, either percutaneously or through open surgery. The cathode and anode can change when using more than two electrodes. For example, FIG. 2A shows a 4 needle electrode setup in which the cathode and anode rotate during the treatment (as used in traditional IRE). If all electrodes are the same size and situated within the tumor, equal and opposite changes in pH will be observed within the target area, effectively cancelling each other out. To prevent this, a grounding ring is utilized to simulate a surface electrode. After IRE treatment of the tumor where the cathode and anode may be rotating, all needle electrodes are connected to the cathode terminal and then the grounding ring is added to the system and connected to the anode (FIG. 2B). The surface electrode sees minimal changes spread over a larger area. IRE controls were evaluated and compared to IRE followed by the optimized pulse parameter found in earlier tests, termed pH-IRE. When comparing IRE control groups to pH-IRE, the modified dose equation (Equation 4) is followed.

In embodiments, when two electrodes were used, the anode and cathode remained constant. When three electrodes were used, each of the three possible electrode pairs was used for one third of the treatment protocol. When four electrodes were used, each of the 6 possible electrode pairs was used for one sixth of the treatment. For groups in which a grounding ring was included, all the electrodes were connected to the cathode and the grounding ring connected to the anode. Energy matched IRE (1000 V at 100 μs) and longer, lower voltage pulse settings (10V at 1 s) per pulse were compared.

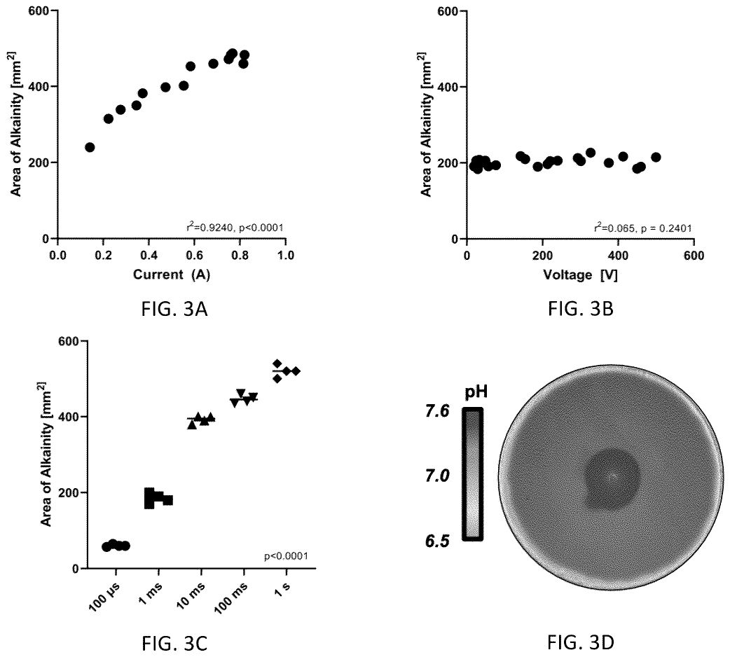

The effects of current, voltage, and pulse width on the pH change in agarose gels are shown in FIGS. 3A-D and in Table 4. Current was increased by adding NaCl to the agar while maintaining a constant voltage, resulting in an increased area of pH change. Voltage was increased while maintaining a constant current by decreasing the NaCl, resulting in a negligible effect on pH. Increasing the pulse width increased the pH effects. FIG. 3A demonstrates how the current (with constant voltage and pulse length) correlates to pH disturbance. FIG. 3B shows that a constant-current treatment (0.100 Amps) (with variations in voltage) within tissue phantoms shows a non-significant correlation to pH change. FIG. 3C shows that pulse width correlates to pH disturbance. FIG. 3D shows a representative image of an agarose tissue phantom containing bromothymol blue following treatment with a 1 s pulse applied at 10 V. Blue represents a pH increase of >1.0. This shows pH is controlled by the current and on-time of the system and is unaffected by voltage.

TABLE 4
Effects of Current, Voltage, and Pulse
Width on pH Change in Agarose Gels.

| Variable 1 | Variable 2 | Constant | Effect on pH |
|---|---|---|---|
| [symbol] Current | [symbol] Conductivity | Voltage, | Increased |
| | | Pulse Width | |
| [symbol] Voltage | [symbol] Conductivity | Current, | Constant |
| | | Pulse Width | |
| [symbol] Pulse Width | [symbol] Voltage, Current | Electric Dose | Increased |

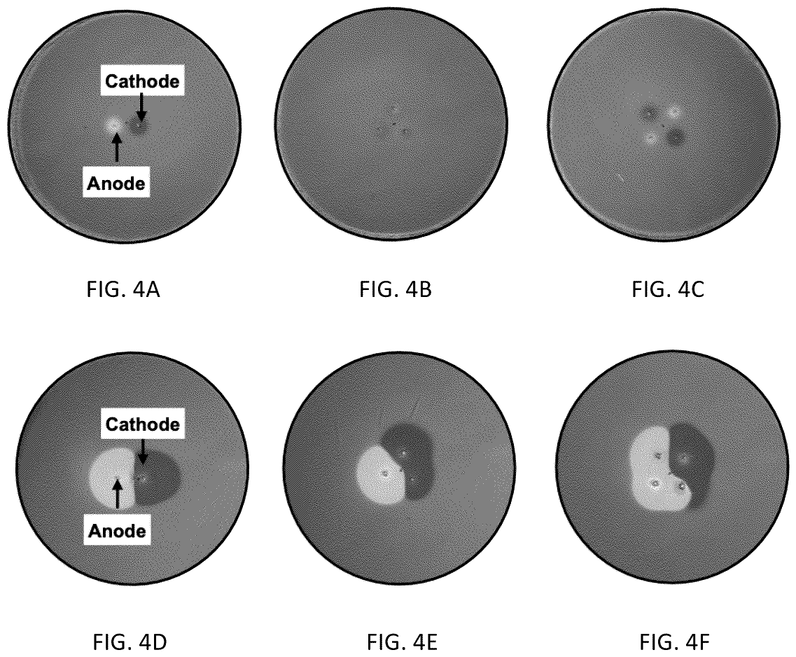

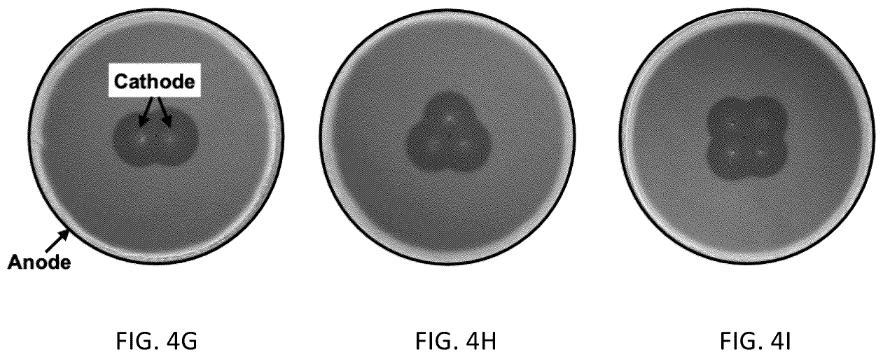

FIGS. 4A-I elaborate on the modulation of PH levels by electrochemical reactions, elucidating the roles of electrode number and geometry. The images show the pH changes in agarose gels using 2-4 needle electrodes for an IRE protocol (200 IRE pulses delivered), a pH-IRE protocol (100 IRE pulses and 100 DC pulses), and a pH-IRE protocol with a surface electrode (100 IRE pulses followed by 100 DC pulses with all needle electrodes connected to the cathode and the surface electrode connected to the anode).

Using a dual electrode arrangement (100 μs pulse length, 1000 V) resulted in distinct, localized pH changes with the pH adjacent to the anode becoming more acidic (yellow) and the pH around the cathode becoming more basic (blue) (FIG. 4A). Using a 3-electrode arrangement and cycling the cathode-anode arrangement (100 μs pulse length, 1000 V) resulted in less clear changes in pH compared to the 2 electrode arrangement (FIG. 4B), whereas the 4 electrode arrangement resulted in 2 regions of increased pH and 2 regions of decreased pH, albeit with smaller regions of pH change compared to the 2 electrode arrangement (FIG. 4C). Altering the pulse delivery parameter to increase pulse length (1 s) at a lower voltage (10 V) led to an increased area of pH change for all electrode arrangements employed (FIGS. 4D-F). When changing the design to incorporate a grounding electrode to serve as the anode, pulse delivery (1 s, 10 V) led to sustained pH increases around the electrodes for all arrangements employed (FIGS. 4G-I).

These data show an appreciable change in pH in a physiological relevant setting that can last for enough time for immune cells to enter the TME before the acidic shield is re-established. It was discovered that pulse duration dictates the electrochemical effect, and that this is a current based phenomenon.

### Area of pH Change

Agarose tissue phantom preparation. A 1% (w/v) agar (ThermoFisher, Waltham, MA) solution was prepared in deionized water (90° C.) and 10% (v/v) bromothymol blue (pH indicator) added (ThermoFisher). To control electrical conductivity, NaCl was added in 0.1% (w/v) increments (final NaCl concentration; 0.1-1.0%) and phantoms were allowed to solidify overnight (room temperature).

Pulse parameters for increasing voltage or current delivery. Pulse width (10 ms), pulse number (100), and frequency (1 Hz) were held constant while voltage or current was increased (i.e. current was maintained with increasing voltage or vice versa) in the NaCl containing phantoms. A single pre-pulse (10 V, 50 μs length) was delivered to each phantom to determine initial resistance and Ohm's law used to determine the change in voltage (or current) required to deliver constant current (or maintain a predetermined voltage).

Increasing Pulse Width. Current (100 mA), voltage (100 V), and frequency (1 Hz) were held constant while pulse width was increased (100 μs−1 s). In doing so, the generator was unable to store sufficient charge to deliver a 1 s pulse at 100 V, so the voltage was dropped to 10 V for the 1 s pulse width and the frequency decreased to 0.5 Hz (allowing a 1 s interval between pulses).

Pulse delivery and pH measurement. Treatments were applied to phantoms using an ECM 830 Square Wave Electroporator (Harvard Apparatus, Holliston, MA). Current and voltage readings were collected using a wideband current monitor (Pearson Electronics, Palo Alto, CA) connected to an oscilloscope (Wavesurfer 3024z, Teledyne LeCroy, Chestnut Ridge, NY). Each treatment was applied using a custom needle-grounding ring electrode configuration. Representative images were captured and area of alkaline pH (pH >7.6=blue) was quantified using ImageJ software (NIH, Bethesda, MD).

Effect of probe number and geometry. Number of electrodes (2-4) and arrangement was varied; When two electrodes were used the anode and cathode remained constant. When three electrodes were used, each of the three possible electrode pairs was used for one third of the treatment protocol. When four electrodes were used, each of the 6 possible electrode pairs was used for one sixth of the treatment. For groups in which a grounding ring was included, all the electrodes were connected to the cathode and the grounding ring connected to the anode. Energy matched IRE (1000 V at 100 μs) and longer, lower voltage pulse settings (10V at 1 s) per pulse were compared.

### Cell Culture

Purified THP-1 monocytes (TIB-202, ATCC, Manassas, VA) were cultured (2×105−8×105 cells/mL) in RPMI-1640 medium supplemented with 10% (v/v) FBS, 1% (v/v) penicillin/streptomycin, and 0.05 mM 2-mercaptoethanol. Differentiation of THP-1 monocytes was induced by the addition of 150 nM phorbol 12-myristate 13-acetate (PMA) (24 hours) followed by replacement with PMA-free culture medium for a further 24 Hrs. To induce M1 activation, differentiated THP-1 macrophages were cultured in the presence of IFN-γ (20 ng/mL) and lipopolysaccharide (LPS; 1 ng/mL) for 48 hours. To induce M2 activation, differentiated THP-1 macrophages were cultured in the presence of interleukin-4 (IL-4; 20 ng/ml) and IL-13 (20 ng/mL) for 48 hours.

### Effect of Altered pH on Macrophage Activity

Cell culture conditions were changed to a CO₂-free incubator and culture medium pH increased from pH 6.5 to pH 8.5 in 1.0 increments using HCl and NaOH titration. After 48 hour exposure to altered pH conditions, THP-1 macrophages were labeled in 100 μL/million cells in eBioscience™ Flow Cytometry Staining Buffer (Thermo Fisher) with 0.1 μL Zombie Aqua™ (BioLegend, San Diego, CA), 2 μL Brilliant Violet 421™ anti-CD-80 antibody (BioLegend, Cat #305222), 1 μL APC anti-CD-206 (MMR) antibody (BioLegend Cat #321110), and 5 μL FITC anti-CD-14 antibody (Abcam Cat #ab28061) prior to fixation and staining using commercial staining and fixation buffers (Thermo Fisher). Positive controls were created by culturing differentiated THP-1 macrophages in 20 ng/mL interferon gamma (IFN-γ) and 1 ng/ml lipopolysaccharide (LPS) for anti-CD-80 and anti-CD-14 and in 20 ng/mL interleukin-4 (IL-4) and 20 ng/ml interleukin-13 (IL-13) for anti-CD-206 in a 5% CO₂ incubator at pH7.4 for 48 hours. Samples were analyzed using a FACSAria Fusion (BD Biosciences, San Jose, CA). 10,000 single-cell events were acquired for samples from each treatment group. Samples were gated for live singlet macrophages that were positive for CD14 and the results analyzed using FlowJo v.10 software (BD, Franklin Lakes, NJ).

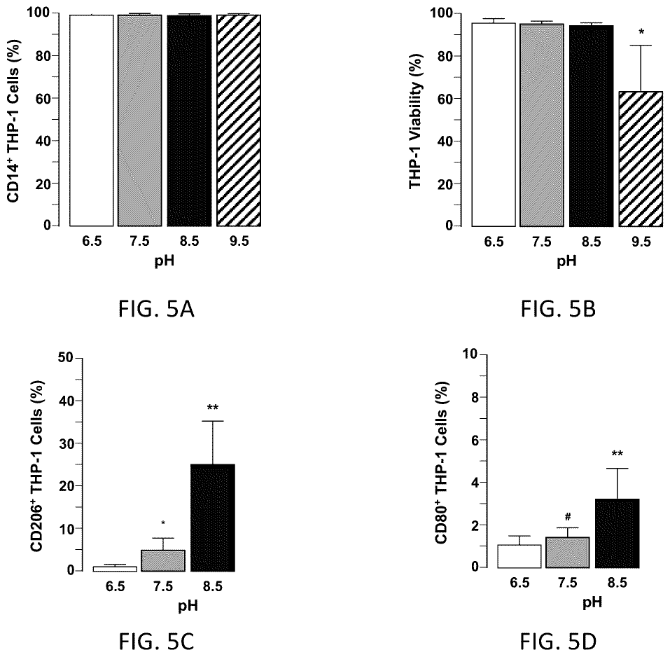

Effect of pH on macrophage viability and activation. FIGS. 5A-D delve into the impact of culture medium pH (altered by the chemicals NaOH and HCl) on the viability and activation of THP-1 macrophages. After gating for live-dead cells, macrophage (THP-1) cell population purity was confirmed (CD14 staining) (FIG. 5A). Increasing culture medium to pH >8.5 led to decreased THP-1 cell viability (FIG. 5B, n=6 independent experiments, p<0.01 pH 9.5 versus all other pH levels tested) and an increased percentage of THP-1 cells staining positive for CD206 (tumor associated macrophage marker) (FIG. 5C, n=6 independent experiments, p<0.0001 pH 8.5 versus pH 7.5 and pH 6.5) and CD80 (marker of M1 activation) (FIG. 5D, n=6 independent experiments, p<0.0001 pH 8.5 versus pH 7.5 and pH 6.5), albeit with a higher percentage of cells staining for CD206 than CD80 at pH 8.5 (FIGS. 5C-D).

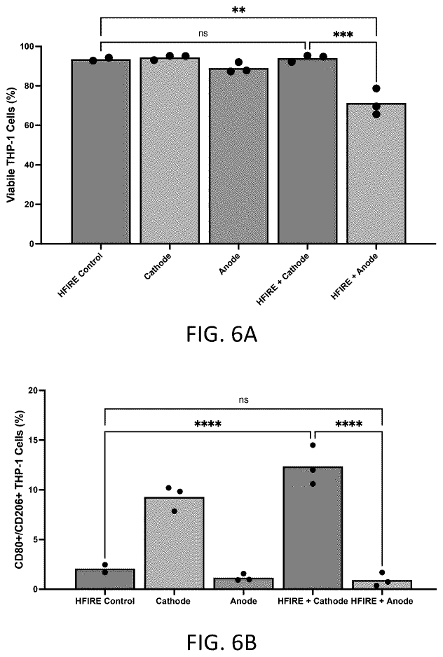

FIGS. 6A-B show the viability and activation of THP-1 macrophages when co-cultured with HEPG2 cells treated with varying combinations of HFIRE, CERs (cathodic electrochemical reactions), and AERs (anodic electrochemical reactions). HFIRE treatment consisted of a 2-5-2-5 μs waveform (2 μs on time, 5 μs delay, 2 μs on time, 5 μs delay) at 1500 V/cm (600 V applied across parallel plates spaced 4 mm apart) for 100 bursts at 1 Hz. CER/AER treatment consisted of 50 s on-time with a 50% Duty Cycle (100 s treatment time—100 μs on/100 μs off over 100 s) at 100 V and 20 mA. The HEPG2 Cells were exposed to HFIRE and then CER or AER and before co-culture with THP-1s in a transwell. Significant decreases in viability of the THP-1 macrophages were noted for the HFIRE+AER group. Significant increases in activation of the THP-1 macrophages were noted for the HFIRE+CER group.

### Porcine Liver Experiments

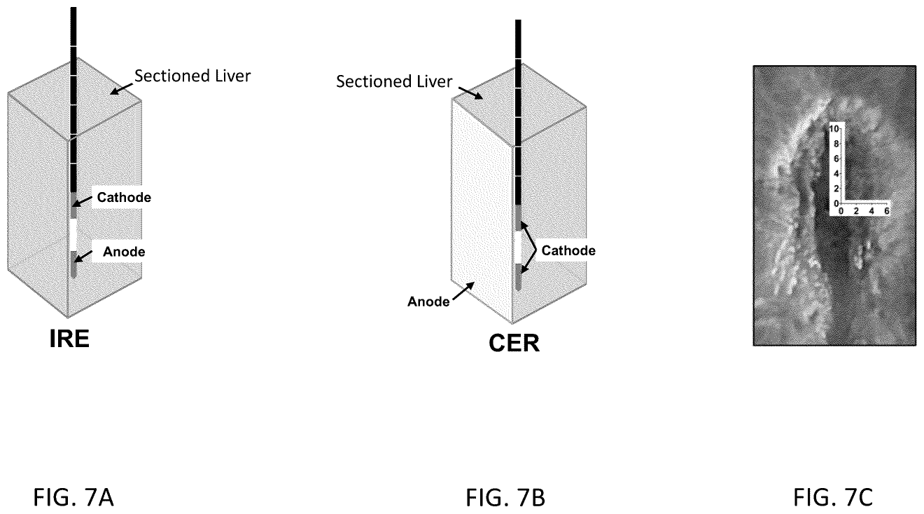

While agar provided convenient ways to visualize the change in pH, it lacks a physiologically relevant environment. To draw conclusions from the previous tests, a porcine liver medium was introduced. FIGS. 7A-B illustrate the methods employed for the delivery of IRE and cathodic electrochemical reactions (CER) to liver tissue. The parameter selection experiments were repeated to confirm the best pulse parameter for magnitude of pH change. Thereafter PH-IRE (i.e. CER-IRE) was compared with an energy matched IRE group to determine area of impact and time of decay. Next, pH measurements were obtained using an Orion™ 8103BNUWP ROSS Ultra™ pH Electrode (ThermoFisher Scientific) along 10 mm points radially and along 15-minute intervals up to 8 hours after treatment.

Porcine liver was obtained immediately following excision from a USDA-approved abattoir and divided into 10×5×3 cm sections to create the geometrical domains required. Tissue collection was deemed exempt from Institutional Animal Care and Use Committee review. An ECM 830 Square Wave Electroporator was employed to deliver IRE pulses using a bipolar needle electrode placed in the center of the liver sample at a depth of 4 cm. During IRE delivery current and voltage readings were collected using a wideband current monitor connected to a Wavesurfer 3024z oscilloscope and pH measurements were made using an Orion™ 8103BNUWP ROSS Ultra™ pH Electrode (ThermoFisher Scientific).

For tissue subject to IRE+CER, a bipolar needle electrode was connected so that the anode and cathode were both initially on the needle electrode (FIG. 7A). IRE pulse delivery consisted of 100 pulses at 1 Hz, 100 μs in length at 1000 V. Immediately after pulse delivery both electrodes on the needle were connected to the cathode and a ground electrode was introduced as the anode (FIG. 7B) for pH/CER treatment (400 pulses, 10 ms in length at 250 V, delivered with a measured current of 2.5 A [taken from the average of first and last pulse]). Immediately following pulse delivery, tissue was sectioned longitudinal to the path of electrode insertion and sequential pH measurements collected in 2 mm increments from the center point of the electrode tip (FIG. 7C). To ensure rapid data collection, measurements were considered symmetrical along each axis and both electrodes were considered as replicates of each other. The pH was also measured at a single point 8 mm radially from the center of the ablation every 15 minutes until pH=6.4 (equal to untreated region).

An ordinary one-way ANOVA and post-hoc Tukey's multi-comparison test was used to analyze differences in macrophage viability and activation and effect of pulse length on pH. Pearson's correlation coefficient was used to analyze effect of increased current and voltage on pH. A p-value <0.05 was considered significant.

### Effect of IRE on Tissue pH in Ex Vivo Liver Tissue

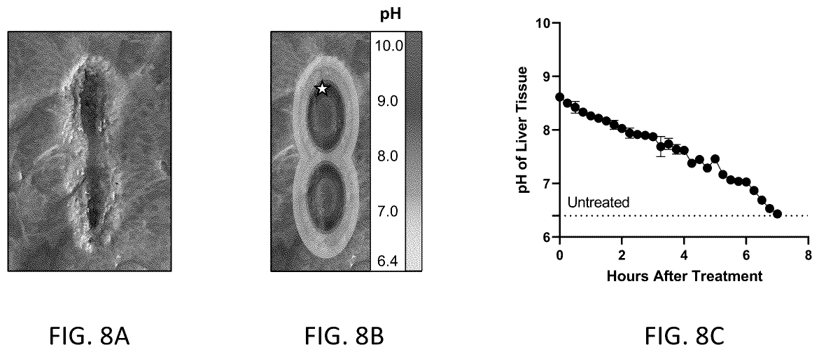

Using a single needle, dual electrode bipolar device ablations were readily detectable in ex vivo porcine liver tissue following pulse delivery (100 pulses, 100 μs length, 1000 V+400 pulses, 10 ms length, 250 V) (FIG. 8A). Measurement of tissue pH immediately following IRE+CER delivery using tissue sectioning demonstrated pH remained significantly elevated up to 12 mm from the center of each needle electrode in the x- and y-planes (FIG. 8B and Table 5). Periodic measurement of pH at 15 min intervals at the margin of basic-neutral tissue reveal pH remained significantly elevated compared to normal tissue up to 7 hours after initial IRE+CER delivery (FIG. 8C).

TABLE. 5
pH of Liver Treated with IRE and CER

| y-axis distance | | x-axis distance | |
|---|---|---|---|
| (mm) | pH | (mm) | pH |
| 0 | 10.5 ± 0.3 | 0 | 10.5 ± 0.3 |
| 2 | 10.2 ± 0.2 | 2 | 9.9 ± 0.3 |
| 4 | 9.5 ± 0.4 | 4 | 8.9 ± 0.1 |
| 6 | 9.2 ± 0.3 | 6 | 8.7 ± 0.2 |
| 8 | 8.9 ± 0.4 | 8 | 8.3 ± 0.1 |
| 10 | 8.6 ± 0.4 | 10 | 7.2 ± 0.3 |
| 12 | 8.3 ± 0.1 | 12 | 6.8 ± 0.5 |
| 14 | 7.5 ± 0.3 | 14 | 6.4 ± 0.0 |
| 16 | 7.0 ± 0.5 | 16 | 6.4 ± 0.0 |
| 18 | 6.4 ± 0.0 | 18 | 6.4 ± 0.0 |

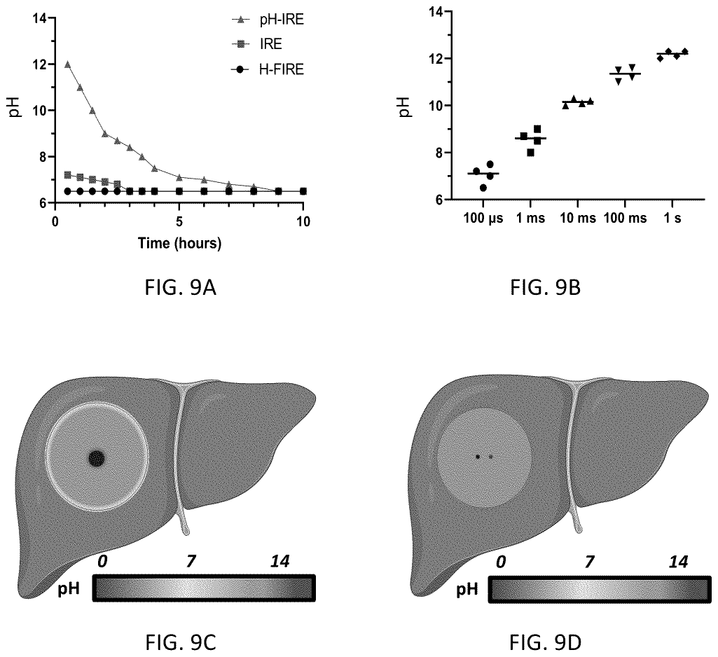

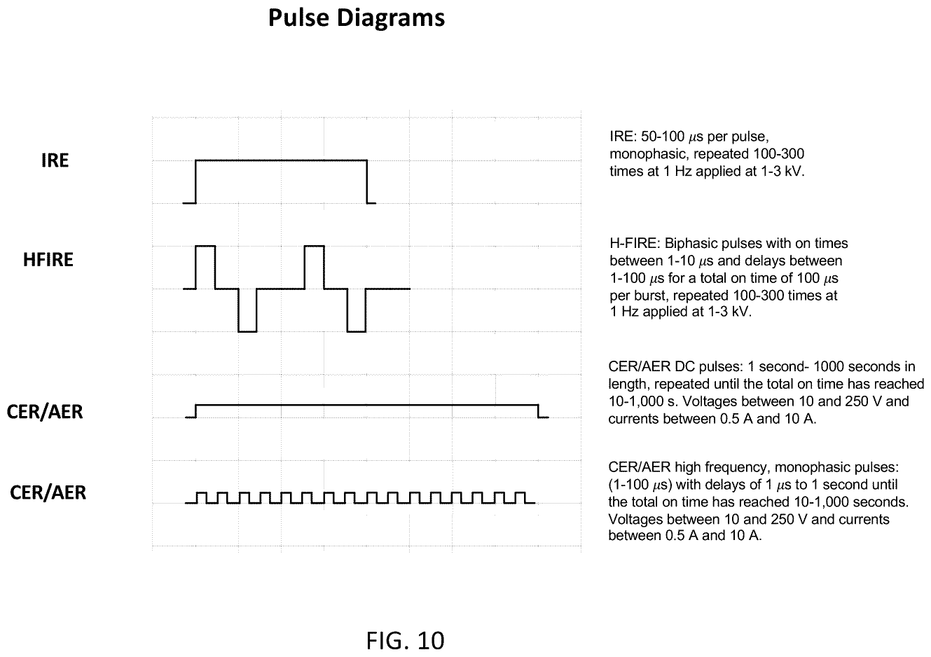

FIG. 9A is a graph showing pH changes over time post pH-IRE, traditional IRE, and High Frequency IRE (HFIRE) treatment. A large increase in pH is observed for several hours post-treatment for the pH-IRE treatment protocol. FIG. 9B is a graph showing pH vs. pulse length for the pH-IRE treatment protocol. The longer the pulse, the greater the increase in pH.

FIG. 9C is an illustration showing the distribution of pH change in an ex-vivo porcine liver using two needles connecting to a cathode plus a distant grounding ring for the anode. FIG. 9D shows a distribution of pH change in an ex-vivo porcine liver using two needles, one connected to the cathode and one connected to the anode.

Currently, much research in the field of electroporation is focused on increasing the area of ablation while limiting thermal effects and muscle contractions. Here, a new method of treatment is described to improve the clinical results of electroporation treatments by utilizing the electrochemical side effects of pulsed electric fields to control pH magnitude, decay rate, and area of impact. The ability to manipulate the pH of the tumor microenvironment may further improve the downstream effects and induce a substantial immune response in electroporation treatments. While pH changes were known to be a side effect of pulsed electric field treatments (Turjanski, P. et al. The Role of pH Fronts in Reversible Electroporation. PloS ONE 6: e17303, 2011), they have not previously been leveraged to overcome the acidic environment of the tumor. In this more targeted approach, local alkaline environments are created to improve the anti-tumor response which is currently limited by tumor acidosis. While this treatment is convenient for IRE as the probes are already within the tumor at the time of treatment, embodiments of the invention include administration of this treatment as a stand-alone treatment or as a combined treatment with a non-electroporation-based treatment such as immunotherapy. Additionally, no plateau of effectiveness is observed as the modified dose equation is manipulated towards increased pulse duration. Further embodiments of the invention include implantation of a source electrode(s) in situ paired with a grounded surface electrode, running long low voltage pulses or even a direct current to continually supply the tumor microenvironment with an alkaline source. There is concern that the surface electrode may result in negative side effects as it creates a negative pH, but this change is negligible when compared to the source electrodes, and the surface electrode could be moved in between treatments if one location became too acidic. This secondary pulsing paradigm brings immunological advantages to a necrotic tumor site filled with inflammatory cytokines post-IRE treatment. This treatment has the advantage of being paired with IRE, a procedure that exists clinically, with a simple addition of a surface electrode.

In embodiments of the invention, the technology described is for experimental and therapeutic applications in which electric fields/currents can be used to change the pH of tissue or other mediums.

In embodiments of the invention, one or more of the IRE electrical pulses have pulse lengths in the range of about 50-200 μs, such as 50 μs, 75 μs, 100 μs, 125 μs, 150 μs, or 175 μs (or any range in between); one or more of the H-FIRE pulses have pulse lengths in the range of about 1-10 μs, such as up to about 1 μs, 2 μs, 3 μs, 4 μs, 5 μs, or 10 μs (or any range in between); one or more of the CER/AER DC pulses have a pulse length of about 1 s to 1,000 seconds (or any range in between), such as 1 s, 2 s, 5 s, 10 s, 20 s, 50 s, 100 s, 150 s, 200 s, 250 s, 300 s, 400 s, 500 s, 600 s, 800 s, or 900 s; and/or one or more of the CER/AER high-frequency, monophasic pulses have a pulse length of about 1-100μ, such as 1 μs, 2 μs, 3 μs, 4 μs, 5 μs, 10 μs, 15 μs, 20 μs, 25 μs, 40 μs, 50 μs, 60 μs, 70 μs, 80 μs, or 90 μs (or any range in between). In embodiments, the pulses, such as the DC pulses, can have a pulse width in the range of up to about 0.1 ms, 0.5 ms, 1 ms, 2 ms, 5 ms, 10 ms, 25 ms, 50 ms, 75 ms, 100 ms, 200 ms, 300 ms, 400 ms, 500 ms, or 750 ms (or any range in between).

In embodiments of the invention, the electrical pulses have no delay between pulses or can be separated by a delay of up to about 10 s, such as up to about 1 ns, 100 ns, 1 μs, 10 μs, 100 μs, 1 ms, 10 ms, 100 ms, 500 ms, 1 s, 2 s, or 5 s (or any range in between). In embodiments of the invention, at least 2 sets of electrical pulses are delivered with a delay between the two sets of pulses, wherein the delay is on the order of seconds, minutes, hours, or days.

In embodiments of the invention one or more electrical pulses are delivered at a voltage of up to about 5,000 V, such as up to about 10 V, 50 V, 100 V, 250 V, 500 V, 750 V, 1,000 V, 1,250 V, 1,500 V, 1,750 V, 2,000 V, 2,500 V, 3,000 V, 3,500 V, 4,000 V, or 4,500 V (or any range in between). In embodiments, the electrodes can be any type of electrode, including needle electrodes, blunt tip electrodes or plate/surface electrodes.

In embodiments of the invention, a total of up to 5,000 electrical pulses are delivered, such as up to about 10, 100, 100-300, 250, 500, 750, 1,000, 1,250, 1,500, 2,000, 2,500, 3,000, 3,500, 4,000, or 4,500 pulses, and so on up to about 1,000,000 pulses (or any range in between).

In embodiments of the invention, one or more of the electrical on times of up to about 1000 s, such as up to about 10 s, 100 s, 500 s, or 1000 s (or any range in between).

In embodiments of the invention, at least 2 sets of electrical pulses are delivered with a delay between the two sets of pulses, wherein the delay is on the order of seconds (such as less than 60 s, 45 s, 30 s, 15 s, 10 s, 5 s, 3 s, 2 s, or 1 s), minutes (such as up to about 60 min, 45 min, 30 min, 15 min, 10 min, 5 min, or 2 min), hours (such as up to about 1 hour, 2 hours, 3 hours, 4 hours, 6 hours, 8 hours, 10 hours, 12 hours, 16 hours, 18 hours, or 24 hours), or days (such as at least 1 day, 2 days, 3 days, 5 days, 10 days, 30 days, or more).

In embodiments of the invention, a total of up to 100,000 electrical pulses are delivered, such as up to about 1,000, 5,000, 25,000, 50,000, 75,000, or 100,000 pulses.

In embodiments of the invention, the IRE pulsing can comprise a) pulses with pulse lengths in the range of about 50-200 μs, such as 50 μs, 75 μs, 100 μs, 125 μs, 150 μs, or 175 μs (or any range in between); b) no delay between pulses or can be separated by a delay of up to about 10 s, such as up to about 1 ns, 100 ns, 1 μs, 10 μs, 100 μs, 1 ms, 10 ms, 100 ms, 500 ms, 1 s, 2 s, or 5 s (or any range in between); c) repeating pulses 100-300 times (or any range in between) at 1 Hz; and/or d) a voltage in the range of 1,000-3,000 V (or any range in between).

In embodiments, H-FIRE pulses can have pulse lengths in the range of about 1-10 μs, such as up to about 1 μs, 2 μs, 3 μs, 4 μs, 5 μs, or 10 μs (or any range in between); delays between pulses of from 1-100 μs, such as 2 μs, 5 μs, 10 μs, 20 μs, 50 μs, or 100 μs (or any range in between); for a total on time of 100 μs/burst; and/or repeated 100-300 times (or any range in between) at 1 Hz at 1,000-3,000 V (or any range in between).

In embodiments, CER can be delivered through high frequency, monophasic pulsing where the pulse width is very short (1-100 μs) (or any range in between) and there are delays between pulses (1 μs to 1 s) (or any range in between) until the total on time has reached 10-1,000 seconds (or any range in between). This can be delivered at voltages between 10 V and 250 V (or any range in between) and currents between 0.5 A and 10 A (or any range in between).

In embodiments, CER can also or in addition be delivered through very long, DC pulses (1 second to 1000 seconds) (or any range in between). In embodiments, a DC pulse can be left on without any delays for several hundred seconds to create the pH change. This can be delivered at voltages between 10 and 250 V (or any range in between) and currents between 0.5 A and 10 A (or any range in between). DC pulses can also be delivered with pulse lengths of 10 ms, 25 ms, 50 ms, 75 ms, 100 ms, or 200 ms (or any range in between).

In embodiments of the invention, the IRE pulsing can be performed in conjunction with CER, either before, during or after the IRE treatment. Such treatment protocols can comprise a) a set of IRE pulses with pulse lengths in the range of about 50-200 μs, such as 50 μs, 75 μs, 100 μs, 125 μs, 150 μs, or 175 μs (or any range in between); b) no delay between pulses or can be separated by a delay of up to about 10 s, such as up to about 1 ns, 100 ns, 1 μs, 10 μs, 100 μs, 1 ms, 10 ms, 100 ms, 500 ms, 1 s, 2 s, or 5 s (or any range in between); c) repeating pulses 100-300 times (or any range in between) at 1 Hz; and/or d) a voltage in the range of 1,000-3,000 V (or any range in between) in combination with: e) a set of CER pulses delivered through high frequency, monophasic pulsing where the pulse width is very short (1-100 μs) (or any range in between) and there are delays between pulses (1 μs to 1 s) (or any range in between) until the total on time has reached 10-1,000 seconds (or any range in between). This can be delivered at voltages between 10 V and 250 V (or any range in between) and currents between 0.5 A and 10 A (or any range in between), or f) CER can also or in addition be delivered through very long, DC pulses (1 second to 1000 seconds) (or any range in between). In embodiments, a DC pulse can be left on without any delays for several hundred seconds to create the pH change. This can be delivered at voltages between 10 and 250 V (or any range in between) and currents between 0.5 A and 10 A (or any range in between). DC pulses can also be delivered with pulse lengths of 10 ms, 25 ms, 50 ms, 75 ms, 100 ms, or 200 ms (or any range in between).

In embodiments of the invention, the H-FIRE pulsing can be performed in conjunction with CER, either before, during or after the H-FIRE treatment. Such treatment protocols can comprise H-FIRE pulses with pulse lengths in the range of about 1-10 μs, such as up to about 1 μs, 2 μs, 3 μs, 4 μs, 5 μs, or 10 μs (or any range in between); delays between pulses of from 1-100 μs, such as 2 μs, 5 μs, 10 μs, 20 μs, 50 μs, or 100 μs (or any range in between); for a total on time of 100 μs/burst; and/or repeated 100-300 times (or any range in between) at 1 Hz at 1,000-3,000 V (or any range in between) in combination with: 1) a set of CER pulses delivered through high frequency, monophasic pulsing where the pulse width is very short (1-100 μs) (or any range in between) and there are delays between pulses (1 μs to 1 s) (or any range in between) until the total on time has reached 10-1,000 seconds (or any range in between). This can be delivered at voltages between 10 V and 250 V (or any range in between) and currents between 0.5 A and 10 A (or any range in between), and/or 2) CER can also or in addition be delivered through very long, DC pulses (1 second to 1000 seconds) (or any range in between). In embodiments, a DC pulse can be left on without any delays for several hundred seconds to create the pH change. This can be delivered at voltages between 10 and 250 V (or any range in between) and currents between 0.5 A and 10 A (or any range in between). DC pulses can also be delivered with pulse lengths of 10 ms, 25 ms, 50 ms, 75 ms, 100 ms, or 200 ms (or any range in between).

In embodiments of the invention, IRE and H-FIRE pulsing can be performed in conjunction with CER, either before, during or after the IRE and H-FIRE treatments. Such treatments can comprise H-FIRE pulses with pulse lengths in the range of about 1-10 μs, such as up to about 1 μs, 2 μs, 3 μs, 4 μs, 5 μs, or 10 μs (or any range in between); delays between pulses of from 1-100 μs, such as 2 μs, 5 μs, 10 μs, 20 μs, 50 μs, or 100 μs (or any range in between); for a total on time of 100 μs/burst; and/or repeated 100-300 times (or any range in between) at 1 Hz at 1,000-3,000 V (or any range in between) and IRE pulses with pulse lengths in the range of about 50-200 μs, such as 50 μs, 75 μs, 100 μs, 125 μs, 150 μs, or 175 μs (or any range in between); b) no delay between pulses or can be separated by a delay of up to about 10 s, such as up to about 1 ns, 100 ns, 1 μs, 10 μs, 100 μs, 1 ms, 10 ms, 100 ms, 500 ms, 1 s, 2 s, or 5 s (or any range in between); c) repeating pulses 100-300 times (or any range in between) at 1 Hz; and/or d) a voltage in the range of 1,000-3,000 V (or any range in between), in combination with: 1) a set of CER pulses delivered through high frequency, monophasic pulsing where the pulse width is very short (1-100 μs) (or any range in between) and there are delays between pulses (1 μs to 1 s) (or any range in between) until the total on time has reached 10-1,000 seconds (or any range in between). This can be delivered at voltages between 10 V and 250 V (or any range in between) and currents between 0.5 A and 10 A (or any range in between), and/or 2) CER can also or in addition be delivered through very long, DC pulses (1 second to 1000 seconds) (or any range in between). In embodiments, a DC pulse can be left on without any delays for several hundred seconds to create the pH change. This can be delivered at voltages between 10 and 250 V (or any range in between) and currents between 0.5 A and 10 A (or any range in between). DC pulses can also be delivered with pulse lengths of 10 ms, 25 ms, 50 ms, 75 ms, 100 ms, or 200 ms (or any range in between).

A factor for CER delivery is the total charge. Charge dictates the total amount of electrons delivered or removed from the system, and these electrons drive the electrochemical reactions at the electrode and tissue interface. The pH change during IRE delivery is proportional to the total charge delivered, where Q is charge, I is current, t is pulse length, and N is pulse number.

$$ΔpH ∝ Q = I * τ * N \qquad ( Equation 5 )$$

### EXAMPLES

These examples are intended to serve as possible ideas and in no way intended to be exhaustive or limit the applications of this technology.

### Example 1: a Combination Therapy of IRE and pH Change

There is an advantage to changing the pH following an Irreversible Electroporation (IRE) treatment. IRE causes an immune response that may be hindered by tumor acidity. For an example IRE treatment involving at least 2 electrodes inserted within the tumor about 2 cm apart with a typical voltage of 3 kV, the electrode exposure is typically 2 cm and 90 3k V pulses are applied with each pulse staying on for about 90 μs and separated by a second between each pulse. Directly following this ˜2 min treatment, the electrodes within the tumor are connected to the same side of the circuit (cathode). A grounding pad is then attached around the abdomen to serve as the anode. DC pulses of 100 ms length are pulsed for 10 pulses at 10 Volts to create a local pH increase of 1-2 units within the tumor. The surface grounding pad can be separated from the skin using a dielectric to create a buffer for the acidic electrolytic effects.

### Example 2: a Stand-Alone Therapy for Solid Tumors to Increase pH and Reverse Immunosuppression

There is an advantage to providing support to the immune system against the tumor microenvironment by changing the pH from acidic to homeostatic or alkaline. Tumors are known to be acidic which can hinder the body's immune response. By using an electrode inserted within the tumor and a grounding pad and dielectric buffer away from the cancer site the pH of the tumor can be increased from acidic to homeostatic. This can be done by administering DC pulses (100 ms length pulsed for 10 pulses at 10 Volts) to create a local pH shift of 1-2 units within the tumor. This pH shift can reverse the immunosuppression of the tumor microenvironment and possibly create an abscopal effect.

### Example 3: Combination Therapy of ECT and pH Change

Electrochemotherapy could be benefited from an increased pH at the site of treatment. Following an ECT treatment, the probes could be connected to the same side of the circuit (cathode) and introduce a surface grounding pad and dielectric buffer to serve as the anode. The increased pH may benefit the success of ECT treatment because of a more robust immune response.

### Example 4: Combination therapy of GET and pH change

Increasing the pH following a gene electro-transfer treatment could improve the body's immune response. GET involves a combination of short pulses to permeabilize membranes and long pulses to electrophoretically force plasmids into the cell. This could be followed by an even longer DC current to generate a local pH shift which could improve the immune response.

### Example 5: Combination Therapy of Pulsed-Field Cardiac Ablation and pH Decrease Through the Anode

Pulsed-field ablation is used to ablate cardiac tissue. Unlike in oncology, an inflammatory response is not desired. After a cardiac ablation, while the electrodes are still inserted within the site, all the in-situ electrodes can be attached to the anode and a distance ground can be introduced to serve as the cathode. The acidic site could suppress inflammatory response which is desirable for cardiac ablation.

### Example 6: Stand-Alone Treatment for Reducing the Inflammatory Response

In situations where acute or chronic inflammation is unnecessary, overacting, or damaging to an individual, it is desirable to decrease the inflammatory response. This can be done by inserting several electrodes into the inflammation site, all connected to the anode. A distant ground can be connected to the body separated by a dielectric buffer connected to the cathode AER treatment can create a local pH decrease of 1-2 units within the inflammation site which can suppress the inflammatory response.

### Example 7: a Combination Therapy of H-FIRE and pH Change

There is an advantage to changing the pH following an High Frequency Irreversible Electroporation (H-FIRE) treatment. H-FIRE causes an immune response that may be hindered by tumor acidity. An H-IRE treatment involves at least 2 electrodes inserted within the tumor about 2 cm apart with a typical voltage of 3 kV. The electrode exposure is typically 2 cm and then 90 3k V pulses are applied with each pulse staying on for about 90 μs and separated by a second between each pulse. Directly following this ˜2 min treatment, the electrodes within the tumor can be connected to the same side of the circuit (cathode). A grounding pad can be attached around the abdomen to serve as the anode. DC pulses of 100 ms length can be pulsed for 10 pulses at 10 Volts to create a local pH increase of 1-2 units within the tumor. The surface grounding pad can be separated from the skin using a dielectric to create a buffer for the acidic electrolytic effects.

### Example 8: The Device can be Left In Situ to Constantly Supply Alkalinity to the Tumor

Because the pH change will reset over time, an effective way to combat the temporal reset is to leave the device in situ at the targeted site to either constantly deliver current or be triggered when the pH dips below a previously determined threshold.

### Example 9: The Technology can be Paired with Additives to Augment the Ablation or the pH Change

Additives to increase ablation area or further change the pH can be paired with this technology. This could include calcium for increased ablation areas, or immunotherapies such as cyclophosphamide for further enhancing immune responses. For increasing the pH this could include sodium bicarbonate, calcium carbonate, or potassium citrate.

### Example 10: Electrodes with Varying Exposure can be Utilized to Develop Alkalinity with Negligible Acidosis

When the cathode has a smaller surface area (1-10%) than the anode the pH effects will be more pronounced at the cathode. This difference in surface area can be tailored to produce the intended alkalinity at the cathode with minimal acidic effects at the anode.

### Example 11: The pH Change can be Administered Prior to, During, or after Ablative Therapy

The pH change can be done before, during, or after the ablative therapy.

### Example 12: This Technology can be Combined with Focal Ablative Therapy

The pH change can be paired with focal ablative therapies outside of electroporation-based modalities, such as but not limited to; radiofrequency ablation, microwave ablation, cryotherapy, and histotripsy.

### Example 13: Utilizing Varying Materials for the Electrode can Further Enhance the Desired Effects

The electrode material will release ions during the electrochemical reactions so picking one that is more inert can be useful to be less harmful while still producing the pH effects. The choice of inert electrodes, such as platinum or palladium, provide the ability to generate pH change through electrolysis with minimal chemical species produced as byproducts. Alternatively, aluminum, steel, or silver chloride electrodes could be leveraged. Also, the surface in contact with the grounding pad could be primed with an alkaline gel to offset the acidosis.

The present invention has been described with reference to particular embodiments having various features. In light of the disclosure provided above, it will be apparent to those skilled in the art that various modifications and variations can be made in the practice of the present invention without departing from the scope or spirit of the invention. One skilled in the art will recognize that the disclosed features may be used singularly, in any combination, or omitted based on the requirements and specifications of a given application or design. When an embodiment refers to “comprising” certain features, it is to be understood that the embodiments can alternatively “consist of” or “consist essentially of” any one or more of the features. Any of the methods disclosed herein can be used with any of the compositions disclosed herein or with any other compositions. Likewise, any of the disclosed compositions can be used with any of the methods disclosed herein or with any other methods. Other embodiments of the invention will be apparent to those skilled in the art from consideration of the specification and practice of the invention.

It is noted in particular that where a range of values is provided in this specification, each value between the upper and lower limits of that range is also specifically disclosed. The upper and lower limits of these smaller ranges may independently be included or excluded in the range as well. The singular forms “a,” “an,” and “the” include plural referents unless the context clearly dictates otherwise. It is intended that the specification and examples be considered as exemplary in nature and that variations that do not depart from the essence of the invention fall within the scope of the invention. Further, all of the references cited in this disclosure are each individually incorporated by reference herein in their entireties and as such are intended to provide an efficient way of supplementing the enabling disclosure of this invention as well as provide background detailing the level of ordinary skill in the art.

1. A method of providing electrical energy therapy comprising:
applying a first set of electrical pulses to a target region sufficient to induce high-frequency irreversible electroporation (H-FIRE) or irreversible electroporation (IRE); and
applying a second set of electrical pulses to the target region insufficient to induce electroporation, and sufficient to change the pH of the target region, by way of a cathode electrode disposed at the target region and an anode electrode disposed away from the target region.

2. The method of claim 1 wherein:
the first set of electrical pulses comprises:
50-200 microsecond IRE pulses of at a voltage ranging from 1,000 V to 5,000 V; or
1-10 microsecond H-FIRE pulses at a voltage ranging from 1,000-5,000 V; and
delivering a second set of electrical pulses capable of changing the pH by increasing or decreasing pH by 1-2 units within the target region;
wherein the first set of pulses is delivered before, after or during the second set of pulses.

3. The method of claim 2, wherein:
the first set of electrical pulses are monopolar IRE pulses delivered at a rate of 1 Hz, with off time between the IRE pulses; or
the first set of electrical pulses are H-FIRE pulses delivered at a burst rate of 1 Hz for a total on time of 100 microseconds per burst.

4. The method of claim 3, wherein the second set of electrical pulses are DC pulses or high-frequency, monophasic pulses.

5. The method of claim 1 comprising:
increasing the pH of a tumor from acidic to homeostatic by applying the second set of pulses as a select number of a select length of DC pulses or high-frequency, monophasic pulses for a select period of time sufficient to raise the pH; and
reversing immunosuppression of a tumor microenvironment.

6. The method of claim 4, wherein:
the second set of pulses comprises DC pulses having a length of from 1-1,000 seconds, which are applied for a total on time in the range of 10-1,000 seconds, and which are applied at a voltage ranging from 10-250 V and a current in the range of 0.5 A to 10 A.

7. The method of claim 1 comprising:
applying the second set of pulses in a manner to increase pH and elevate an immune response or in a manner to decrease pH and suppress inflammatory response.

8. The method of claim 1, wherein:
the first set of electrical pulses are IRE pulses:
in the range of 100-300 pulses, delivered with off time between the pulses;
delivered at a voltage in the range of 1,000-3,000 V; and
having a length of from 50-100 microseconds; and
the second set of electrical pulses:
comprises monophasic, high-frequency pulses of from 1-100 microseconds with delays between pulses of from 1 microsecond to 1 second, or comprises DC pulses with a length of from 1-1,000 seconds;
is applied for a total on time in the range of 10-1,000 seconds; and
is applied at a voltage of 10-250 V and a current in the range of 0.5-10 A.

9. The method of claim 1, wherein:
the first set of electrical pulses:
comprises a number of H-FIRE pulses in the range of 100-300 pulses, each having a length of from 1-10 microseconds; and
is applied at a voltage of 1,000-3,000 V; and
the second set of electrical pulses:
comprises monophasic, high-frequency pulses or comprises DC pulses; and
is applied at a voltage of 10-250 V and a current in the range of 0.5-10 A.

10. The method of claim 1, wherein:
the second set of pulses is delivered in a manner to increase the pH from acidic to basic.

11. The method of claim 1, wherein the second set of pulses:
comprises pulses having a pulse length of 1 microsecond to 10 seconds;
comprises 10-100 pulses; and
is applied at a voltage in the range of 10-100 V.

12. The method of claim 1, wherein the change in pH is an increase or decrease in pH of 1-2 units.

13. The method of claim 1, wherein the first and/or second set of electrical pulses are AC pulses, DC pulses, or a combination of AC and DC pulses.

14. The method of claim 1, wherein:
the applying of the first and second set of electrical pulses comprises applying a plurality of electrical pulses to a tissue of the target region, wherein the plurality of electrical pulses is capable of causing electroporation and electrolysis of the tissue.

15. A method comprising:
administering a plurality of electrical pulses to a tumor by way of a cathode electrode disposed at the tumor site and an anode electrode disposed at another site, with the surface area of the anode relatively larger than the surface area of the cathode, wherein the plurality of electrical pulses is administered in a manner capable of:
changing the pH of the tumor microenvironment; and/or
causing a desired immune response; and/or
improving immune cell infiltration; and/or
controlling immune cell phenotype.

16. A method comprising:
connecting one or more or multiple probes to the cathode of a pulse generator;
connecting a ground electrode to the anode of the pulse generator; and
delivering a plurality of electrical pulses to a treatment area;
wherein at least one pulse of the plurality of electrical pulses is capable of causing irreversible electroporation of at least some cells in the treatment area; and
wherein at least one pulse of the plurality of electrical pulses is capable of changing the pH of the treatment area by isolating alkaline reactions generated at the treatment area thereby reversing an acidic microenvironment of the treatment area.

17. The method of claim 16, wherein the ground electrode is a distant ground electrode that is a surface electrode.

18. The method of claim 16, wherein at least one pulse of the plurality of electrical pulses is applied at a voltage capable of changing the pH, but below a voltage threshold capable of nerve excitation and/or irreversible electroporation.

19. The method of claim 1, further comprising measuring the change in pH.
